(19) United States
(12) Patent Application Publication
(10) Pub. No.: US 2006/0068416 A1
(43) Pub. Date: Mar. 30, 2006

(54) Devices coated with substances which mediate the adhesion of biological material

(75) Inventors: Hermann Schluesener, Tuebingen (DE); Hans-Peter Wendel, Balingen (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Eberhard-Karls-Universitaet Tuebingen Universitaetsklinikum, Tuebingen (DE)

(21) Appl. No.: 11/151,123

(22) Filed: Jun. 13, 2005

Related U.S. Application Data
(63) Continuation of application No. PCT/EP03/13989, filed on Dec. 10, 2003.

(30) Foreign Application Priority Data
Dec. 17, 2002 (DE) 102 58 924.0

(51) Int. Cl. C12Q 1/68; G01N 33/53; C12M 1/34

(52) U.S. Cl. 435/6; 435/7.1; 435/287.2

(57) ABSTRACT

Devices having at least one surface which comes into contact with tissues and/or fluids of the human or animal body. These surfaces are at least partially coated with aptamers, which mediate the adhesion of biological material.

### CROSS-REFERENCE TO RELATED APPLICATION

This is a continuation of copending International Patent Application PCT/EP2003/013989 filed Dec. 10, 2003, the entire disclosure of which is incorporated herein by reference.

### BACKGROUND OF THE INVENTION

1. Field of the Invention

The invention relates to devices which includes at least one surface which comes into contact with tissues and/or fluids of the human or animal body and which is at least partially coated with substances which mediate the adhesion of biological material.

2. Related Prior Art

A large number of such devices, and the methods for producing them, are disclosed in the prior art.

Devices comprising coated surfaces are of particular importance when the devices come into contact with human tissue or blood as is the case, for example, in connection with an extracorporeal blood circulation system or in connection with blood vessel prostheses.

In the case of an extracorporeal blood circulation, which has to be used, for example, in connection with operations on the open heart or in connection with dialysis, blood comes, at least briefly, into contact with synthetic surfaces, for example of tubes, pumps, oxygenators, etc. This contact can trigger coagulation and clumping reactions in the blood, which reactions can produce, inter alia, life-threatening thromboses, inflammatory reactions and the formation of biofilm (bacterial colonization). In the case of heart and/or lung support systems, it is also even possible to envisage contact extending over several weeks in connection with catheter and blood vessel orifices. Storage systems for blood components, or contact lenses, for example, can also come into contact with human or animal tissue over a relatively long period of time.

Furthermore, devices which come into contact with human blood or tissue also include implants which are inserted into the human body permanently or for a given period of time. Examples of devices which are inserted permanently are artificial heart valves, artificial hip or knee joints, heart pacemakers and tooth implants, while examples of devices which are inserted transiently are plates and screws which are made of artificial (metal, ceramic or plastic) or animal material which is as immunologically inert as possible. The devices furthermore include blood vessel prostheses, conduits, patches, catheters, artificial bladders, etc. which can in principle consist of any polymeric plastics, metals, alloys, textiles or natural products (chitosan, bacterial cellulose, etc.) or else of other degradable materials.

In addition, prostheses, for example in the form of stents, are frequently employed in vascular surgery, with these prostheses being fabricated from a variety of plastics or metals. Since these prostheses are exogenous structures, inflammatory reactions, encapsulation of the foreign structure by proliferation of the surrounding tissue as a rejection reaction, and complications in blood coagulation, and restenoses, can be observed repeatedly.

It is not only in connection with the abovementioned applications that there is a necessity to coat surfaces such that they exhibit good biocompatibility towards blood or other tissue parts. Within its widest sense, biocompatibility means the compatibility of substances with living biological material (bones, tissues, blood, organs, etc.). In order to avoid complications such as coagulation, proliferation, inflammatory reactions and rejection reactions, devices which come into contact with blood, tissues, etc. are nowadays coated with biocompatible materials. The materials which are used today are intended to behave inertly in the body and not to have any significant influence on the metabolism.

The approach of colonizing implants in vitro with the patient's own cells is, for example, known in the prior art. As part of this concept, autologous tissue is worked-up and cultured in vitro, and an implant which is “matched” to the patient is produced in combination with suitable matrices. This implant is then introduced into the body of the donor and matures in the recipient into a tissue which is as naturalistic as possible. This is intended to prevent implants from being recognized by the body as being foreign and then being rejected.

A disadvantage of producing implants which are colonized with cells in vitro by means of tissue engineering is that the colonization of these implants or devices is extremely elaborate and has to be carried out under the strictest sterile conditions in order to be able to achieve a sufficiently high degree of success. In the case of such implantable devices, cells have first of all to be isolated from the patient in whom such an implant is to be used. The cells have then to be cultured, and replicated, on the implant in question and, finally, the implant has to be introduced surgically into the body of the patient. All these steps make this method extremely time-consuming and expensive.

It has furthermore also been frequently observed that, while the cells have replicated on the surface of the implants, they have at the same time lost many properties as a result of dedifferentiation. Thus, for example, isolated cartilage cells scarcely form any cartilage substance or else only form a cartilage substance which is atypical. Intestinal epithelial cells or kidney tubule cells can no longer take up substances in the known manner and have substantially poorer transport and sealing functions.

It is furthermore known in the prior art to use cell-binding peptides or proteins to mediate the adhesion of cells to a surface which is coated with these peptides/proteins.

The scientific literature describes, in particular, the use of integrin-specific peptides, or of laminin derivatives, for coating implants (see, for example, Kantlehner et al., “Surface coating with cyclic RGD peptides stimulates osteoblast adhesion and proliferation as well as bone formation”, Chembiochem 18:107-114, 2000; Tamura et al., “Coating of titanium alloy with soluble laminin-5 promotes cell attachment and hemidesmosome assembly in gingival epithelial cells: potential application to dental implants”, J. Periodontal Res. 32(3): 287-294, 1997; Kaushal S. et al., “Functional small-diameter neovessels created using endothelial progenitor cells expanded ex vivo”, Nat. Med. 7(9): 1035-1040, 2001).

DE 197 55 801 furthermore discloses implants which, for the purpose of stimulating the adhesion of body cells in a targeted manner, contain peptides which possess sequences which recognize the binding sites on the integrin receptors of cells. In this connection, these peptides are arranged in a defined pattern on the surface of the implant.

However, using peptides suffers from the disadvantage that the construction and synthesis of these peptides are very elaborate, thereby consequently also making the production of devices which are coated with these peptides elaborate and expensive.

### SUMMARY OF THE INVENTION

Against this background, the invention provides devices which include a substance mediating the adhesion of biological material and which can be produced inexpensively and without any great consumption of time, with the devices at the same time exhibiting good biocompatibility properties and being able to be colonized with cells and/or proteins in a simple manner.

Accordingly, the invention provides a device in which aptamers are the substances mediating the adhesion of biological material.

In this way, the objective of the invention is achieved in full.

It is possible to coat surfaces with aptamers such that biological material can be immobilized at the surfaces by way of these aptamers.

Aptamers are high-affinity RNA or DNA oligonucleotides or polynucleotides which, because of their specific spatial structure, possess a high affinity for a target molecule.

Herein, “biological material” refers to any target molecules which are bound by way of aptamers and includes, for example, other nucleic acids, proteins or protein fragments, lipoproteins, glycoproteins and protein complexes and also small organic molecules or even cells and microorganisms, such as viruses.

Aptamers are frequently even more specific than antibodies and exhibit antigen-binding properties which are comparable to those of antibody fragments. Due to their possessing of a relatively large and flexible surface, they can potentially interact with more target molecules than can smaller molecules.

Large quantities of oligonucleotides of a very wide variety of sequences and secondary structures can be generated enzymically by means of “SELEX” (systematic evolution of ligands by exponential enrichment). Oligonucleotides having a high affinity for a target molecule are then picked out from this pool and concentrated. If the primary structure of such an oligonucleotide is known, the oligonucleotide can then also be synthesized chemically. An exemplary method for obtaining suitable aptamers is described, for example, in DE 100 19 154.

The aptamers which have been found can then also be modified using suitable techniques such that they are protected and do not lose their activity in the biological environment, for example are not digested by nucleases. Protective mechanisms which are suitable for this purpose are adequately disclosed in the prior art and include, for example, LNA (locked nucleic acids) technologies using furanose (see, for example: Wahlestedt et al., “Patent and nontoxic antisense oligonucleotides containing locked nucleic acids”, Proc. Natl. Acad. Sci., USA 97(10): 5633-5638, 2000) or the Spiegelmer® technology from the company Noxxon (Berlin, Germany).

It is now possible, according to the invention, to provide devices whose surfaces are at least partially coated with particular aptamers by way of which native biological material can be immobilized at the surface.

An advantage of such an aptamer coating is that this coating is stable and sterilizable, thereby making it possible to produce aptamer-coated devices inexpensively. As compared with peptides, another advantage is that, while peptides frequently lose their. activity as a result of the sterilization, oligonucleotides, that is aptamers, are extremely stable.

In this connection, it is not always necessary to coat all the surfaces of the devices; on the contrary, it is only necessary, and to some extent also desirable, to coat particular surfaces on the device with the aptamers or to coat different surfaces of a device with different aptamers. In this way, it is possible to achieve the situation where biological material only binds to the surfaces which are coated with the aptamers or where different biological materials bind to surfaces comprising different aptamers.

This is advantageous, for example, in the case of stents, blood vessel prostheses, blood vessel apertures, ports or conduits, which can be coated differently on their inner surface, which comes into contact with blood, for example, as compared with their outer surface, which comes into contact with the tissue surrounding the stent and which is intended to grow into this tissue.

In the case of the devices which are coated in accordance with the invention, biological material from blood, tissues, organs or other sources is fixed, resulting in the generation of autologous functional interfaces, layers or cell formations which are consequently no longer recognized by the body as being foreign and which take on the functional physiological properties of the particular site of use or organ.

The invention provides the before-mentioned device, wherein the aptamers are nucleic acid molecules which comprise at least one of the sequences SEQ ID No. 1 to SEQ ID No. 17 from the enclosed sequence listing.

It can be demonstrated that nucleic acid molecules which contain at least one of the above nucleotide sequences recognize and bind native biological material. Nucleic acid molecules which contain one of the listed sequences are distinguished, according to the inventors' findings, by a high degree of specificity for the biological material employed.

It will be understood, therefore, that a nucleic acid molecule which comprises at least one of the nucleotide sequences SEQ ID No. 1 to 17 from the enclosed sequence listing is likewise encompassed by the invention.

Preference is furthermore given to the nucleic acid molecule being a nucleic acid molecule having one of the nucleotide sequences SEQ ID No. 1 to SEQ ID No. 17 from the enclosed sequence listing.

Nucleic acid molecules having the disclosed sequences have proved to be particularly suitable for binding biological material.

In this connection, preference is given to the aptamers being attached to the surface of the device either directly and/or by way of a linker molecule.

In this connection, “linker molecule” or “linker” refers to any substance which can be used to attach an aptamer on the surface.

In this connection, preference is given to the linker molecule being N-succinimidyl-3-(2-pyridyldithio) propionate and/or a PEG block copolymer which is, for example, linear or stellar.

It has already been demonstrated that it was possible to use the substance N-succinimidyl-3-(2-pyridyldithio)propionate in connection with immobilizing a regulator of the complement system on particular surfaces of biomaterials (see Anderson et al., “Binding of a model regulator of complement activation (RCA) to a biomaterial surface: surface-bound factor H inhibits complement activation”, Biomaterials 22: 2435-2443, 2001). Using this linker did not impair the biological activity of the regulator. PEG block copolymers, which have likewise proved to be suitable linkers, are comprehensively described, for example, in Tirelli et al. “Poly(ethylene glycol) block copolymers”, Biotechnol. 90(1): 3-15, 2002.

Furthermore, the aptamers can, in principle like any nucleotides, be attached (for example after coupling to amino or biotin groups at the 3′ or 5′ end) to the surface of the devices by way of suitable linker molecules or spacers. Methods for immobilizing oligonucleotides are described, for example in “Immobilisierung von Oligonucelotiden an aminofunktionalisierte Silizium-Wafer [Immobilization of oligonucleotides on amino-functionalized silicon wafers]”(U. Haker, Chem. Dissertation, Hamburg, 2000), with 1,4-phenylenediisothiocyanate, inter alia, being employed in this connection. Other important covalent methods for modifying surfaces are described in the dissertation “Miniaturisierte Affinitätsanalytik-Ortsaufgelöste Oberflächenmodifikationen, Assays und Detektion [Miniaturized affinity analysis-site-resolved surface modifications, assays and detection]” (I. Stemmler, Chem. Dissertation, Tubingen, 1999) and in the Hermanson et al. publications “Immobilized affinity ligand techniques” (Academic Press, San Diego, 1992) and “Bioconjugate Techniques” (Academic Press, San Diego, 1996). Thus, SiO₂, TiO₂, —COOH, HfO₂, —Au, —Ag, N-hydroxysuccinimide, —NH2, epoxide, maleimide, acid hydrazide, hydrazide, azide, diazirine, benzophenone, and others, can, for example, be used as functional anchors in couplings together with a variety of coreactants.

Photolinking constitutes another method for immobilizing oligonucleotides on surfaces. In this method, the NH₂-coupled oligonucleotide (aptamer) is first of all provided with what is termed a photolinker molecule (e.g. anthraquinone) which can subsequently, under UV activation, enter into photochemical reactions with a synthetic surface and thereby bind the oligonucleotide covalently to the surface. Kits and substances for carrying out this method can be obtained, for example, from the company Exiqon (Vedbaek, Denmark) under the names AQ-Link™ and DNA Immobilizer™.

Preference is furthermore given to the biological material comprising cells which are selected from the group containing stem cells, epithelial cells, endothelial cells, muscle cells, fibroblasts, osteoblasts, keratinocytes, astrocytes, retinocytes, Langerhans' cells, hepatocytes, cardiomyocytes, chondrocytes or chondroblasts or their precursor cells.

It can be demonstrated that endothelial cells are bound by way of certain selected aptamers, in particular by way of those aptamers which comprise a nucleic acid molecule having the nucleotide sequence SEQ ID No. 1 to SEQ ID No. 17. In this way, aptamer-coated surfaces of stents, for example, can bind endothelial cells as a result of which the stents can be adapted optimally to the tissues lining the blood vessels.

Preference is furthermore given to the biological material comprising proteins which are selected from the group comprising plasma proteins, membrane proteins, receptor proteins, integrins, enzymes, transducers, signal substances and messenger substances, as well as fragments thereof.

Preference is given, in particular, to the proteins employed being fibronectin, laminin, vitronectin, thrombomodulin or high molecular weight kininogen, or fragments thereof.

Thus, immobilized aptamers can be used to bind contact phase proteins (high molecular weight kininogen, HMWK; inter alia) to foreign surfaces, thereby making it possible to avoid adsorption of fibrinogen. It is furthermore possible to immobilize inhibitors/regulators possessing key functions within the hemostaseologic cascade reactions (AT-III, C1-esterase-INH, complement factor H, thrombomodulin, plasminogen, inter alia), as well as VEGFR-1, VEGFR-2 (KDR), Tie-2, CD133 and CD43 on a surface.

This embodiment according to the invention can, for example, be employed in extracorporeal blood circulation systems in which blood comes into contact with foreign tube surfaces. These devices can accordingly be coated with aptamers, for example, which mediate the adhesion of substances which prevent blood coagulation and/or inflammatory reactions.

This binding of cells and/or proteins to the aptamer-coated surface advantageously creates an autologous surface structure which avoids a host-versus-graft response in connection with implants, for example, and thus avoids consequential implantation costs which frequently arise in connection with such reactions.

In another embodiment, the device according to the invention can additionally be coated with growth factors. This embodiment has the advantage that, for example, precursors of the abovementioned cells, such as endothelial progenitor cells (EPC) are bound to the surfaces by way of aptamers and, by means of specific growth factors, which act as inducers, are differentiated into full-blown endothelium. In this connection, these growth factors can, for example, also be immobilized on the surface of the device by way of aptamers (coimmobilization). In this connection, preference is given to the growth factors being selected from the group comprising platelet-derived growth factor (PDGF), vascular endothelial growth factor (VEGF), colony-stimulating factor (CSF), epidermal growth factor (EGF), nerve growth factor (NGF), fibroblast growth factor (FGF), and/or growth factors from the TGF superfamily series. Examples of growth factors from the TGF (transforming growth factor) superfamily are BMPs (bone morphogenetic proteins) such as BMP-2 and BMP-7.

Thus, vascular prostheses which have been pretreated in this way can, for example, immediately after having been implanted, bind EPCs, from the blood circulating through them, to the surface and endothelialize the prosthesis material within a very short period of time.

Preference is furthermore given to an embodiment in which the aptamers exhibit an enzymic activity, preferably DNAzyme activity.

This has the advantage of providing a coating which is at least partially enzymically active. If, for example, aptamers possessing DNAzyme activity are employed specifically, these aptamers degrade the mRNA which is recognized by the DNAzymes. This can be advantageous, for example, in connection with regulating the production of Egr-1 protein. The Egr-1 protein is a protein which is required in connection with the growth of smooth musculature. Using vascular prostheses which have been coated in this way prevents, for example, the growth of blood vessels. Furthermore, such enzymically active aptamers can be used, for example, to regulate blood coagulation cascades.

In another embodiment, preference is given to using, as the surface, a material which is selected from the group comprising polytetrafluoroethylene, polystyrene, polyurethane, polyester, polylactide, polyglycolic acid, polysulfone, polypropylene, polyethylene, polycarbonate, polyvinyl chloride, polyvinyl difluoride, polymethyl methacrylate, polyethylene terephthalate, ePTFT, texin (polyether-polyurethane) or copolymers thereof, nylon, silanized glass, ceramic or metal, in particular titanium, or mixtures thereof.

Furthermore, the materials employed can also be nanomaterials and/or nanomaterials which are composed of DNA building blocks and which contain a certain percentage of aptamers.

These materials have proved to be of value in the fields which are concerned with, for example, tissue engineering or vascular surgery generally and are employed in a variety of embodiments.

In this connection, the shape of the surface can be selected at will.

Devices which are coated in accordance with the invention include, for example, any apparatuses or tubes which are employed in an extracorporeal blood circulation, as well as catheters and blood vessel orifices, contact lenses, storage systems for blood components, and other surfaces. In this connection, preference is given, according to the invention, to the device being an implant.

Suitable implants are, in particular, artificial hearts, heart valves, vascular prostheses, artificial organs, stents, artificial hips, bones, tendons, ligaments, joints, cartilage, dental implants, artificial corneas, skin, intestine, intraocular lenses, acellularized organs, vascular implants, etc., in which only the original supporting structure is still present, and many others. In the case of these surfaces, there is a need to bring about selective cell adhesion.

The devices which are coated in accordance with the invention are either coated with cells in vivo, that is in situ, i.e. directly in the patient in whom the autologous tissue then forms on the implanted device, or else ex vivo or in vitro.

The devices which are coated in accordance with the invention can furthermore be used as bioreactors for isolating, and subsequently propagating, particular cell types for the purpose of producing particular substances or as an organ replacement (liver, pancreas, etc.).

The invention encompasses the use of a nucleic acid molecule including one of the nucleotide sequences 1 to 17 from the accompanying sequence listing.

It is possible to use the nucleic acid molecules according to the invention to immobilize endothelial progenitor cells selectively.

In a another embodiment, these nucleic acid molecules are furthermore coupled to a diagnostic agent and/or therapeutic agent.

These modified nucleic acid molecules or aptamers enjoy a multiplicity of advantages as compared with the monoclonal antibodies which are customarily used in diagnosis. Because of the sequence-determined formation of secondary structures, the repertoire of potentially binding ligands is substantially greater than the immune repertoire which is available for preparing monoclonal antibodies.

Furthermore, aptamers can be provided substantially more rapidly and more inexpensively. Millions of potential ligands can be analyzed within three to four weeks.

Fluorescent compounds, e.g. fluorescein isothiocyanate (FITC), biotin, dioxygenin and their derivatives, enzyme labels, infrared labels and gelatinizing agents are particularly suitable for use as diagnostic agents within the context of a diagnostic method.

It is possible to use the claimed nucleic acid molecules for coating surfaces, and thereby to use them directly as what might be termed “trapping molecules,” herewith making it possible to immobilize the biological target structure in situ within tissues and/or fluids.

The invention also provides a method for coating devices comprising at least one surface which comes into contact with tissues and/or fluids and which is at least partially coated with substances which mediate the adhesion of biological material, with the method including the following steps:

providing aptamers which mediate the adhesion of biological material, and, binding the aptamers from step a) to the surface of a device.

In this connection, preference is given to using, as aptamers, nucleic acid molecules which comprise at least one of the nucleotide sequences SEQ ID No. 1 to SEQ ID No. 17 from the enclosed sequence listing.

The devices which are prepared using this method can, for example, be used directly in an extracorporeal blood circulation or as an implant or the like.

Other advantages ensue from the figures and the following example.

The features which are mentioned above, and those which are still to be explained below, can be used not only in the combination which is in each case specified but also in other combinations, or on their own, without departing from the scope of the present invention.

Exemplary embodiments of the invention are depicted in the drawing and are explained in more detail in the following description.

### BRIEF DESCRIPTION OF THE DRAWINGS

FIGS. 1a+b show the characterization of the claimed nucleic acid molecules by means of flow cytometry.

### DESCRIPTION OF PREFERRED EMBODIMENTS

Selecting Aptamers which are Directed Against EPC (Endothelial Progenitor Cells)

EPCs derived from Lewis rat bone marrow were cultured in commercially available selection and propagation media.

Oligonucleotides (aptamers) which bind to EPCs were selected from a library (synthetic oligonucleotides, MWG Biotech, Germany) of DNA oligonucleotides which are known to bind to intercellular regulatory (transcription) factors. In this connection, the same method was employed, while using EPCs, as has already been described in DE 100 19 154 and in the publication “Systematic evolution of a DNA aptamer binding to rat brain tumor microvessels: selective targeting of endothelial regulatory protein pigpen” (Blank et al., J. Biol. Chem. 2001; 276(19):16464-8).

The sequences and designations of the identified oligonucleotides which bind EPC are shown in table 1 below:

| | | | |
|---|---|---|---|
| FACS No. | Oligonucleotides | Sequences | Results |

| | | | |
|---|---|---|---|
| Control 1 | Cells | | neg. |
| Control 2 | SEL III 11-1 | ctg ttg gac att caa aag ac | neg. |
| a) | 4 cpg FITC | tcg tcg ttt tgt cgt ttt gtc gt | pos. |
| c) | Trirep 3 | ccg ccg ccg ccg ccg ccg ccg | pos. |
| d) | Trirep 5 | gcg gcg gcg gcg gcg gcg gcg | pos. |
| e) | Trirep 6 | cgg cgg cgg cgg cgg cgg cgg | pos. |
| f) | Trirep 8 | ttc ttc ttc ttc ttc ttc ttc | pos. |
| g) | Trirep 10 | tta ggt tag gtt agg tta ggt tag g | pos. |
| h) | Cpgoligo 1 | gct aga cgt tag cgt | pos. |
| i) | Cpgoligo 1 control | gct aga gct tag gct | pos. |
| j) | Cpgoligo 2 | gat tgc ctg acg tca gag ag | pos. |
| k) | Cpgollgo 3 | ttc atg acg ttc ctg atc gt | pos. |
| l) | Cpgoligo 3 control 1 | tcc atg act ttc ctc agg tt | pos. |
| m) | Cpgoligo 3 control 2 | tcc atg agc ttc ctg atg ct | pos. |
| n) | Cpgoligo 4 control | tgc tgc ttt tgt gct ttt gtg ctt | pos. |
| o) | dnazegr 1 | ccg cgg cca ggc tag cta caa cga cct gga cga t | pos. |
| p) | aptzymegr 1 | att gtg gtt ggt agt ata cat ttt tcc gcg gcc agg cta gct aca acg | pos. |
| | | acc tgg acg at | |
| q) | Soxs-2-78-113 | ctt taa tgc ggg gta att tct ttt cca taa tcg c | pos. |
| r) | Cysk-2-66-106 | tta ttt ccc ttc tgt ata tag ata tgc taa atc ctt act t | pos. |

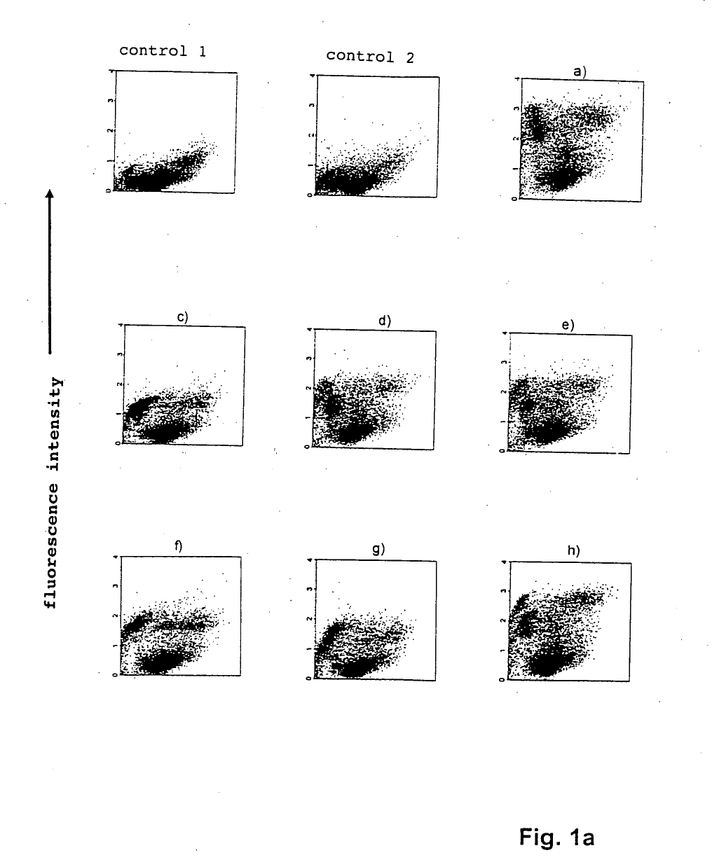

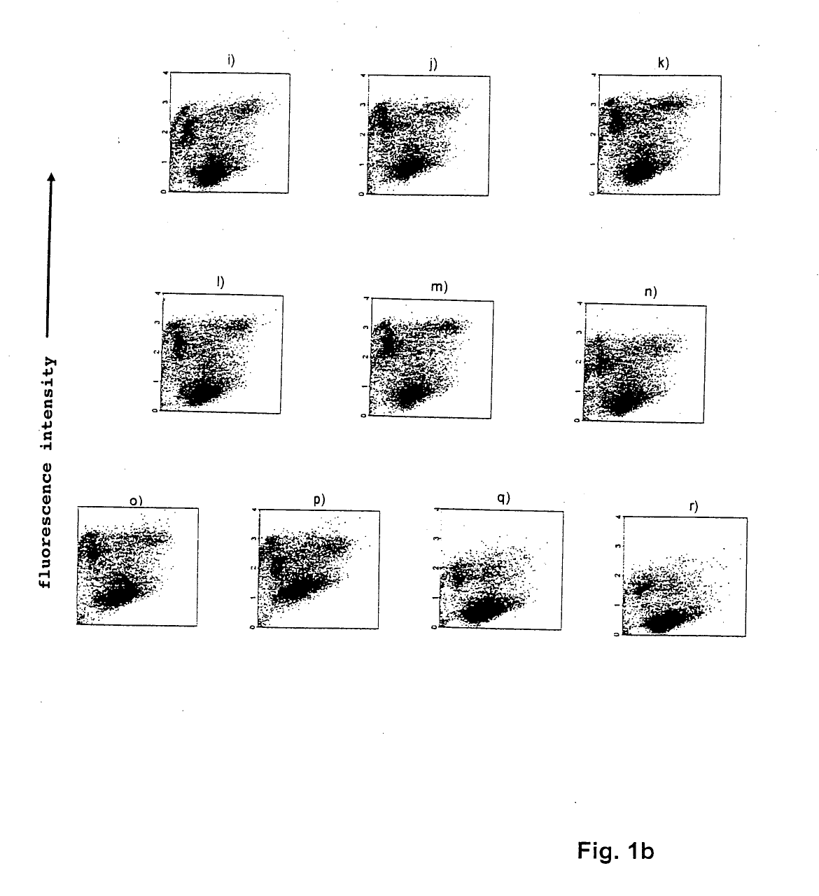

The oligonucleotides were labeled with FITC (fluorescein isothiocyanate) and their binding to EPCs was detected by means of flow cytometry (FACS). The results of these analyses are shown in FIGS. 1a and 1b and summarized in table 1. In table 1, “pos.” means that the cells (EPCs) bind to the FITC-labeled aptamers. The oligonucleotide SEL III 11-1, which has been demonstrated not to bind to EPC, was used as the control. The oligonucleotides a) to r) from table 1 were tested cytometrically and the results of these analyses are depicted in plots a) to r) in FIGS. 1a and b, which plots correspond respectively to the oligonucleotides a) to r) employed.

As can be seen in plots a) to r) in FIGS. 1a and 1b, the identified oligonucleotides without exception give rise to positive binding reactions. Oligonucleotides o) and p) were identified as being DNAzymes.

The sequences 1 to 17 listed in the sequence listing correspond to oligonucleotides a) to r) as given in table 1.

A flow-through cell which is positioned under a fluorescence microscope is used for measuring immobilization processes. The flow-through cell contains aptamer-coated carrier material (linker: photolinker, AQ photochemistry, Exiqon, Denmark). The cell is perfused with cell suspensions, protein solutions, plasma, blood or other relevant biological solutions. In this connection, the target (protein, cell, etc.), is fluorescence-labeled. The rate at which the targets become attached (captured) can, for example, be recorded using a video camera.

Instead of using a flow-through cell, it is also possible to use a commercially available ELISA plate which is coated with the aptamer. The target can be quantified using a labeled antibody in accordance with standard ELISA techniques.

1. A device comprising at least one surface which comes into contact with tissues and/or fluids of the human or animal body and which is at least partially coated with substances which mediate the adhesion of biological material, wherein the substances are aptamers.

2. The device as claimed in claim 1, wherein the aptamers are nucleic acid molecules which comprise at least one of the sequences SEQ ID No. 1 to SEQ ID No. 17 from the enclosed sequence listing.

3. The device as claimed in claim 1, wherein the aptamers are nucleic acid molecules having at least one of the nucleotide sequences SEQ ID No. 1 to SEQ ID No. 17 from the enclosed sequence listing.

4. The device as claimed in claim 1, wherein the aptamers are attached to the surface directly and/or by way of a linker molecule.

5. The device as claimed in claim 4, wherein the linker molecule is N-succinimidyl-3-(2-pyridyldithio)propionate and/or a PEG block copolymer.

6. The device as claimed in claim 1, wherein the biological material comprises cells which are selected from the group consisting of: stem cells, epithelial cells, endothelial cells, muscle cells, fibroblasts, osteoblasts, keratinocytes, astrocytes, retinocytes, Langerhans' cells, hepatocytes, cardiomyocytes, chondrocytes and chondroblasts and their precursor cells.

7. The device as claimed in claim 1, wherein the biological material comprises proteins which are selected from the group consisting of: plasma proteins, membrane proteins, receptor proteins, integrins, enzymes, transducers, signal substances and messenger substances, and fragments thereof.

8. The device as claimed in claim 7, wherein the proteins are selected from the group consisting of: fibronectin, laminin, vitronectin, thrombomodulin, high molecular weight kininogen, AT-III, C1-esterase-INH, complement factor H, plasminogen, VEGFR-1, VEGFR-2 (KDR), Tie-2, CD133, CD43, and fragments thereof.

9. The device as claimed in claim 1, further comprising growth factors.

10. The device as claimed in claim 9, wherein the growth factors are selected from the group consisting of: platelet-derived growth factor (PDGF), vascular endothelial growth factor (VEGF), colony-stimulating factor (CSF), epidermal growth factor (EGF), nerve growth factor (NGF), fibroblast growth factor (FGF) and growth factors from the transforming growth factor (TGF) superfamily.

11. The device as claimed in claim 1, wherein the aptamers exhibit an enzymic activity.

12. The device as claimed in claim 11, wherein the aptamers exhibit a DNAzyme activity.

13. The device as claimed in claim 1, wherein the surface comprises a material which is selected from the group consisting of: polytetrafluoroethylene, polystyrene, polyurethane, polyester, polylactide, polyglycolic acid, polysulfone, polypropylene, polyethylene, polycarbonate, polyvinyl chloride, polyvinyl difluoride, polymethyl methacrylate, polyethylene terephthalate, ePTFT, texin (polyether-polyurethane) and copolymers thereof, nylon, silanized glass, ceramics, metals, and mixtures thereof.

14. The device as claimed in claim 1, wherein the device is an implant.

15. A nucleic acid molecule which comprises at least one of the nucleotide sequences SEQ ID No. 1 to SEQ ID No. 17 from the enclosed sequence listing.

16. A nucleic acid molecule as claimed in claim 15, wherein the molecule is coupled to at least one of a diagnostic agent and a therapeutic agent.

17. A method for producing devices comprising at least one surface which comes into contact with tissues. and/or fluids of the human or animal body and which is at least partially coated with substances which mediate the adhesion of biological material, comprising the following steps:
a) providing aptamers which mediate the adhesion of biological material,
b) binding the aptamers from step a) to the surface of a device.

18. The method as claimed in claim 17, wherein the aptamers employed are nucleic acid molecules which comprise at least one of the nucleotide sequences SEQ ID No. 1 to SEQ ID No. 17 from the enclosed sequence listing.

19. The method as claimed in claim 18, wherein the aptamers employed are nucleic acid molecules having at least one of the nucleotide sequences SEQ ID No. 1 to SEQ ID No. 17 from the enclosed sequence listing.

20. The device as claimed in claim 1, wherein the surface comprises titanium.


SEQUENCE LISTING (17 sequences)

SEQ ID NO: 1 - DNA, 23 bp, Artificial sequence
  Synthetic aptamer
tcgtcgttttgtcgttttgtcgt

SEQ ID NO: 2 - DNA, 21 bp, Artificial sequence
  Synthetic aptamer
ccgccgccgccgccgccgccg

SEQ ID NO: 3 - DNA, 21 bp, Artificial sequence
  Synthetic aptamer
gcggcggcggcggcggcggcg

SEQ ID NO: 4 - DNA, 21 bp, Artificial sequence
  Synthetic aptamer
cggcggcggcggcggcggcgg

SEQ ID NO: 5 - DNA, 21 bp, Artificial sequence
  Synthetic aptamer
ttcttcttcttcttcttcttc

SEQ ID NO: 6 - DNA, 25 bp, Artificial sequence
  Synthetic aptamer
ttaggttaggttaggttaggttagg

SEQ ID NO: 7 - DNA, 15 bp, Artificial sequence
  Synthetic aptamer
gctagacgttagcgt

SEQ ID NO: 8 - DNA, 15 bp, Artificial sequence
  Synthetic aptamer
gctagagcttaggct

SEQ ID NO: 9 - DNA, 20 bp, Artificial sequence
  Synthetic aptamer
gattgcctgacgtcagagag

SEQ ID NO: 10 - DNA, 20 bp, Artificial sequence
  Synthetic aptamer
ttcatgacgttcctgatcgt

SEQ ID NO: 11 - DNA, 20 bp, Artificial sequence
  Synthetic aptamer
tccatgactttcctcaggtt

SEQ ID NO: 12 - DNA, 20 bp, Artificial sequence
  Synthetic aptamer
tccatgagcttcctgatgct

SEQ ID NO: 13 - DNA, 24 bp, Artificial sequence
  Synthetic aptamer
tgctgcttttgtgcttttgtgctt

SEQ ID NO: 14 - DNA, 34 bp, Artificial sequence
  Synthetic aptamer
ccgcggccaggctagctacaacgacctggacgat

SEQ ID NO: 15 - DNA, 59 bp, Artificial sequence
  Synthetic aptamer
attgtggttggtagtatacatttttccgcggccaggctagctacaacgacctggacgat

SEQ ID NO: 16 - DNA, 34 bp, Artificial sequence
  Synthetic aptamer
ctttaatgcggggtaatttcttttccataatcgc

SEQ ID NO: 17 - DNA, 40 bp, Artificial sequence
  Synthetic aptamer
ttatttcccttctgtatatagatatgctaaatccttactt